(19) United States
(12) Patent Application Publication
(10) Pub. No.: US 2007/0004986 A1
(43) Pub. Date: Jan. 4, 2007

(54) Electronic device and module

(75) Inventors: Seppo Korkala, Kempele (FI); Markku Karjalainen, Kempele (FI)

Correspondence Address: HOFFMANN & BARON, LLP, 6900 JERICHO TURNPIKE, SYOSSET, NY, 11791, US

(73) Assignee: Polar Electro Oy

(21) Appl. No.: 11/453,552

(22) Filed: Jun. 15, 2006

(30) Foreign Application Priority Data
Jul. 4, 2005 (FI) 20055385

(51) Int. Cl. A61B 5/02

(52) U.S. Cl. 600/500; 343/742; 340/870.31; 340/573.1; 368/10

(57) ABSTRACT

An electronic device and a module for installation into an electronic device. The solution presented includes at least one set of induction means for generating a magnetic field for an electronic circuit employing the magnetic field; and a communications unit coupled to the at least one set of induction means; wherein the induction means and the communication unit are configured to implement a wireless communications link based on the magnetic field.

### CROSS-REFERENCE TO RELATED APPLICATION

This application claims priority based on Finnish Patent Application No. 20055385, filed on Jul. 4, 2005, which is incorporated herein by reference.

### FIELD OF THE INVENTION

The invention relates to an electronic device and to a module for an electronic device.

### BRIEF DESCRIPTION OF THE RELATED ART

The aim in the design of electronic devices is small-sized electronic circuits and effective component density to save space and costs. A typical bulky component is a macroscopic inductance coil, a plurality of which may be comprised by an electronic device for different purposes. Thus, it is useful to study techniques for achieving space saving in an electronic device.

### SUMMARY OF THE INVENTION

The object of the invention is to provide an electronic device and a module for an electronic device so as to achieve space saving in an electronic device.

As a first aspect of the invention there is provided an electronic device comprising: at least one set of induction means for generating a magnetic field for an electronic circuit employing the magnetic field; a communications unit coupled to said at least one set of induction means; and the induction means and the communication unit being configured to implement a wireless communications link based on the magnetic field.

As a second aspect of the invention there is provided a module for installation into an electronic device, the module comprising: at least one set of induction means for generating a magnetic field for at least one electronic circuit of the electronic device employing the magnetic field; a communications unit coupled to said at least one set of induction means; and the induction means and the communication unit being configured to implement a wireless communications link based on the magnetic field.

Preferred embodiments of the invention are described in the dependent claims.

The invention is based on employing the same induction means both for generating a magnetic field for an electronic circuit and for implementing a wireless communications link based on the magnetic field.

The electronic device and the module of the invention bring forth a plurality of advantages. An advantage is the achievement of space saving and cost saving, since the wireless communications link does not require a separate induction coil structure.

### BRIEF DESCRIPTION OF THE DRAWINGS

In the following, the invention will be described in more detail in connection with preferred embodiments with reference to the accompanying drawings, in which

FIG. 1 shows a first example of an embodiment of an electronic device;

FIG. 2 shows an example of a receiver in a communications unit;

FIG. 3 shows an example of a transmitter in a communications unit;

FIG. 4 shows a second example of an embodiment of an electronic device;

FIG. 5 shows an example of a signal timing diagram; and

FIG. 6 shows a third example of an embodiment of an electronic device.

### DETAILED DESCRIPTION OF THE PREFERRED EMBODIMENTS

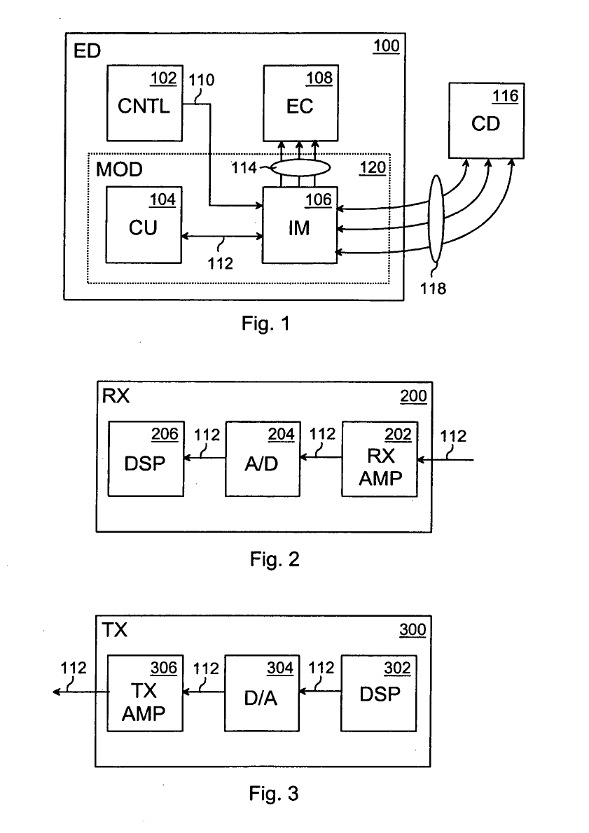

With reference to the example of FIG. 1, an electronic device (ED) 100 comprises induction means (IM) 106, a communications unit (CU) 104 coupled to the induction means 106, an electronic circuit (EC) 108, and a controller (CNTL) 102 coupled to the electronic circuit 108.

The induction means 106 induce a magnetic field 114 as a result of electric current 110 introduced into the induction means 106. The induction means 106 may comprise an induction coil, for example. In an embodiment, the induction means 106 also comprise a magnetic core in connection with the induction means 106, such as an iron core.

The controller 102 is an electronic circuit that supplies electric current 110 to the induction means 106 in a manner required by the electronic circuit 108.

The magnetic field 114 may be directed to the electronic circuit 108 by selecting the direction of the induction means 106 in such a manner that the magnetic flux of the magnetic field 114 is in the desired direction in the electronic circuit 108. If a magnetic core is in use, the magnetic flux may be introduced into the electronic circuit 108 via the magnetic core.

The electronic circuit 108 is a circuit that employs the magnetic field 114 for instance for converting voltage or for generating mechanical energy from electric energy. The induction means 106 may have common structures, such as the magnetic core, with the electronic circuit 106.

The communications unit 104 and the induction means 106 together implement a wireless communications link 118 that may be established with the electronic device 100 and a wireless communications device (CD) 116 supporting the wireless communications link 118. The communications unit 104 and the induction means 106 communicate with one another via a communications signal 112.

In an embodiment, the electronic device 100 is part of a performance measurement system that registers a user's performance and/or activity. The performance measurement system may comprise a plurality of communications devices that communicate with each other by means of wireless data transfer and may comprise measurement sensors making measurements from the user and/or the environment. As an example may be mentioned a system, wherein the electronic device 100 is a central unit of the performance measurement system and the communications device 116 is a measurement sensor, such as a sensor measuring the electrocardiogram, for example. The central unit of the performance measurement system may be a wrist device to be installed in a user's wrist, for example. In an embodiment, the electronic device is a wrist device of a heart rate monitor.

The communications link 118 is based on a variable electromagnetic field generated and/or detected by the induction means 106, the magnetic component of the field being detected in a receiver. The coverage of the communications link 118 based on the magnetic component is typically a few meters with a transmission power in the order of milliwatts. The frequency employed by the communications link 118 may be some kilohertz, such as 5 kHz, for example. However, the solution presented is not restricted to said frequency or frequency range, but it may be any frequency achievable with induction coil structures.

In an embodiment, the communications unit 104 comprises a receiver. In this case, an electromagnetic field generated by the communications device 116 is typically directed to the induction means 106, the field transferring information wirelessly from the communications device 116 to the electronic device 100 and inducing voltage in the poles of the induction means 106. In this case, the communications signal 112 is typically voltage, the information contained by the communications link 118 being encoded in the levels of the voltage.

In the example of FIG. 2, a receiver (RX) 200 typically comprises a receiver amplifier (RX AMP) 202, an analog-to-digital converter (A/D) 204 coupled to the receiver amplifier 202, and a digital signal processor 206 (DSP) coupled to the analog-to-digital converter 204.

The receiver amplifier 202 receives a communications signal 112, amplifies the communications signal 112, and supplies the amplified communications signal 112 to the analog-to-digital converter 204. The analog-to-digital converter 204 converts the communications signal 112 from an analog form into a digital form and supplies the digital communications signal 112 to the digital signal processor 206. The digital signal processor 206 processes the communications signal 112 and may execute processes on the basis of information contained by the communications signal 112.

In an embodiment, the communications unit 104 comprises a transmitter. In this case, the communications signal 112 includes electric pulses that are generated by the communications unit 104 and into which information is coded. The electric pulses are supplied into the induction means 106, wherein the electric pulses induce an electromagnetic field that generates the communications link 118.

In the example of FIG. 3, a transmitter 300 comprises a digital signal processor (DSP) 302, a digital-to-analog converter (D/A) 304 coupled to the signal processor 302, and a transmitter amplifier (TX AMP) 306 coupled to the digital-to-analog converter 304.

The digital signal processor 302 generates the communications signal 112 as a result of a process executed in the electronic device 100, for example, and supplies the communications signal 112 to the digital-to-analog converter 304. The digital-to-analog converter 304 converts the digital communications signal 112 into an analog form and supplies the analog communications signal 112 to the transmitter amplifier 306. The transmitter amplifier 306 amplifies the communications signal 112 and supplies the communications signal 112 to the induction means 106.

With further reference to FIG. 1, as an aspect of the invention there is presented a module (MOD) 120 comprising at least induction means 106 and communications means 104. The module 120 may be manufactured separately from the electronic device 100 and installed into the electronic device 100 at the manufacturing stage of the electronic device 100. The module may comprise other components, too, such as an electronic circuit 108 and/or a controller 102.

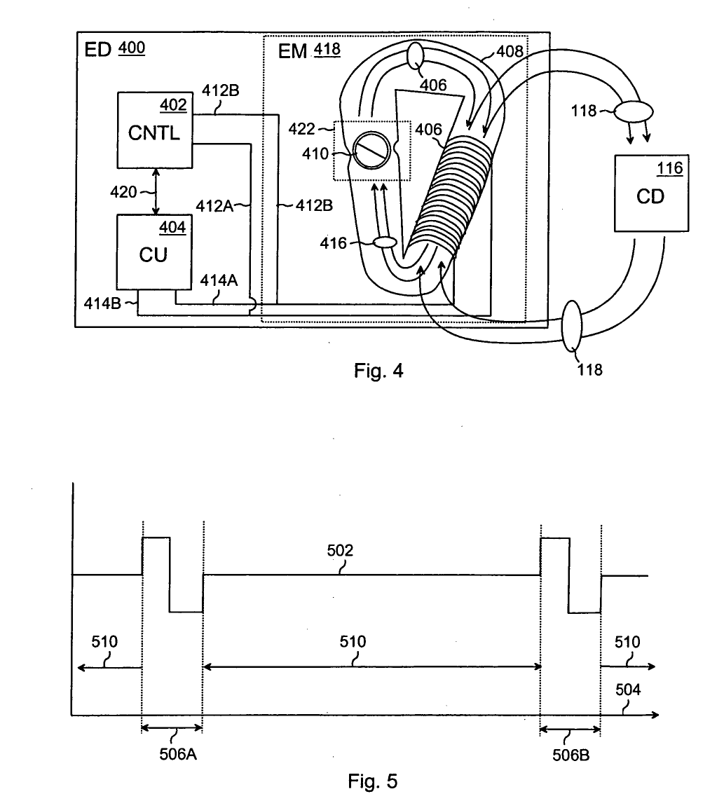

With reference to the example of FIG. 4, in an embodiment, the electronic circuit of an electronic device 400 comprises an electric motor (EM) 418 that converts the energy comprised by a magnetic field 416 into mechanical energy. In this case, the induction means is a solenoid 406 of the electric motor 418, and a magnetic core 408 may be arranged inside the solenoid. The magnetic core 408 may constitute part of the frame of the electric motor 418.

A controller 402 supplies supply signals 412A, 412B to the solenoid 406, and the signals transfer electric power to the electric motor.

In the example of FIG. 4, the magnetic core 408 constitutes a magnetically closed circuit comprising a stator part 422. The stator part 422 constitutes a structure that surrounds a rotor 410 and wherein the direction of the magnetic field 416 changes in time causing the rotor 410 to rotate. The rotational energy of the rotor 410 may be conducted by means of a power transmission mechanism to a destination of use, such as a pointer on a clock. The rotor 410 may comprise a permanent magnet that is oriented on the basis of the direction and strength of the magnetic flux generated by the stator part 422.

In the example shown in FIG. 4, the controller 402 is a microcomputer unit, for example, whose supply signals 412A, 412B are digital pulses.

In the example shown in FIG. 4, the communications unit 404 may comprise a receiver 200 according to FIG. 2 and/or a transmitter 300 according to FIG. 3.

With reference to FIG. 5, an embodiment of the operation of the electric motor 418 will be studied, wherein the electric motor 418 operates as a step motor. The step motor is the power source of the electromechanical watch of a wrist device, for example.

In an embodiment, the electronic device 100 is a watch.

FIG. 5 shows a voltage curve 502 illustrating the voltage difference between the supply signals 412A, 412B of the controller 402 of FIG. 4 as a function of time shown on a time axis 504.

The voltage curve 502 comprises operational cycles 506A, 506B, whose distance determines the distance of the steps of the step motor operating as the electric motor 418, for example. If the step motor is the step motor of a watch, the distance may be for instance one second, a multiple of a second or an even-divided part of minutes. The duration of the operational cycle 506A, 506B may be a few milliseconds, for example.

FIG. 5 also shows a communications cycle 510, during which the wireless communications link 118 is active.

In an embodiment, the electronic circuit 108 and the communications unit 104 of FIG. 1 are configured to operate non-simultaneously. Referring further to the example of FIG. 4, the non-simultaneity may be implemented by the exchange of synchronization information between the communications unit 404 and the controller 402.

In an embodiment, the communications unit 404 generates synchronization information 420 and supplies the synchronization information 420 to the controller 402. On the basis of the synchronization information 420, the controller 402 may time the supply signals 412A, 412B of the electric motor in such a manner that the electric motor 418 operates when the communications unit 404 is passive. In this case, the synchronization information 420 may comprise information on the timing of the communications cycle 510, for example.

In an embodiment, the controller 402 generates synchronization information 420 and supplies the synchronization information 420 to the communications unit 404. On the basis of the synchronization information 420, the communications unit 404 may time the communications signal 414A, 414B in such a manner that the communications unit 404 operates when the electric motor 402 is passive. In this case, the synchronization information 420 may comprise information on the timing of the operational cycles 506A, 506B, for example.

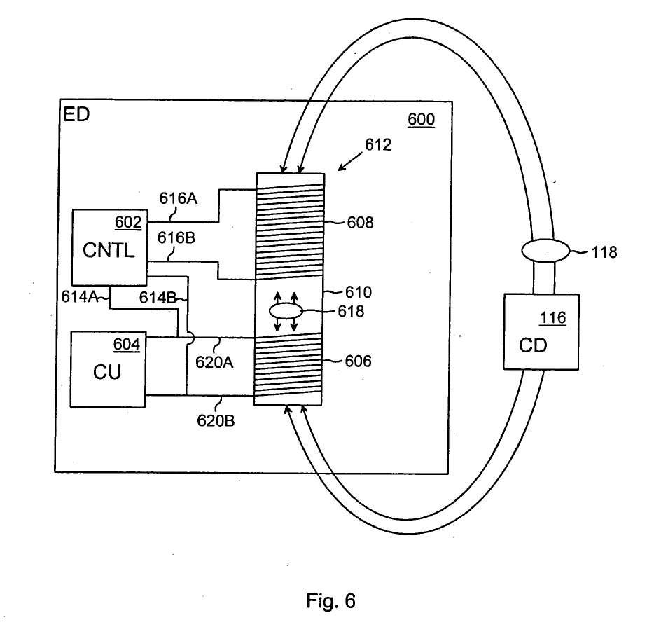

Referring to the example of FIG. 6, in an embodiment, an electronic circuit 600 comprises a transformer (TR) 612. The transformer 612 comprises a primary coil 606, a magnetic core 610, and a secondary coil 608. In an embodiment, the induction means 106 of FIG. 1 operate as the primary coil 606. In another embodiment, the induction means 106 of FIG. 1 operate as the secondary coil 608. The magnetic core 610 is an iron core, for example. The structure and operation of a transformer are generally known to those skilled in the art and they are therefore not described in more detail in this connection.

The controller 602 of FIG. 6 supplies alternating voltage 614A, 614B to the primary coil 606. A magnetic field 618 is generated in the secondary coil 608, and the field induces alternating voltage 616A, 616B in the secondary coil 608, the voltage being supplied to the controller 602. The controller 602 may comprise a rectifier for rectifying the alternating voltage 616A, 616B.

The communications unit 604 exchanges communications signals 620A, 620B corresponding to the communications signal 112 of FIG. 1 with the primary coil 606 and/or the secondary coil 608.

In the example shown in FIG. 6, the communications unit 604 may comprise a receiver 200 according to FIG. 2 and/or a transmitter 300 according to FIG. 3.

In an embodiment, the communications unit 604 comprises a filter circuit for filtering the alternating voltage 614A, 614B of the transformer of the filter circuit from the communications signal 620A, 620B. The filter circuit may be a high-pass filter, for example, which damps the low-frequency alternating voltage 614A, 614B and 616A, 616B in such a manner that the low-frequency alternating voltage 614A, 614B and 616A, 616B is denied access to the receiver 200 and/or the transmitter 300 of the communications unit 604.

Although the invention is described herein with reference to the example in accordance with the accompanying drawings, it will be appreciated that the invention is not to be so limited, but it may be modified in a variety of ways within the scope of the appended claims.

1. An electronic device comprising:
at least one set of induction means for generating a magnetic field for an electronic circuit employing the magnetic field; and
a communications unit coupled to said at least one set of induction means, wherein the induction means and the communication unit are configured to implement a wireless communications link based on the magnetic field.

2. The electronic device of claim 1, wherein the electronic circuit comprises an electric motor for converting energy comprised by the magnetic field into mechanical energy, and wherein the induction means are adapted to operate as a solenoid of the electric motor.

3. The electronic device of claim 1, wherein the electronic circuit comprises a transformer, and wherein the induction means are adapted to operate as an induction coil of a transformer.

4. The electronic device of claim 1, wherein the electronic circuit and the communications unit are configured to operate mutually non-simultaneously.

5. The electronic device of claim 1, wherein the electronic device is part of a performance measurement system that registers a user's performance, and wherein the induction means and the communications unit are configured to implement the wireless communications link based on the magnetic field between a communications device of the performance measurement system.

6. A module for installation into an electronic device, the module comprising:
at least one set of induction means for generating a magnetic field for at least one electronic circuit of the electronic device employing the magnetic field; and
a communications unit coupled to said at least one set of induction means, and wherein the induction means and the communication unit are configured to implement a wireless communications link based on the magnetic field.

7. The module of claim 6, wherein the electronic circuit comprises an electric motor for converting energy comprised by the magnetic field into mechanical energy, and wherein the induction means are adapted to operate as a solenoid of the electric motor.

8. The module of claim 6, wherein the electronic circuit comprises a transformer, and wherein the induction means are adapted to operate as an induction coil of a transformer.

9. The module of claim 6, wherein the electronic circuit and the communications unit are configured to operate mutually non-simultaneously.

10. The module of claim 6, wherein the electronic device is part of a performance measurement system that registers a user's performance, and wherein the induction means and the communications unit are configured to implement the wireless communications link based on the magnetic field between a communications device of the performance measurement system.

11. The electronic device of claim 1, wherein the electronic device is a watch.

12. The electronic device of claim 1, wherein the electronic device is a wrist device of a performance measurement system.

13. The module of claim 6, wherein the electronic device is a watch.

14. The module of claim 6, wherein the electronic device is a wrist device of a performance measurement system.
